(19) United States
(12) Patent Application Publication
(10) Pub. No.: US 2017/0152066 A1
(43) Pub. Date: Jun. 1, 2017

(54) METHOD FOR MANUFACTURING PACKAGED MEDICAL DEVICE

(71) Applicant: TERUMO KABUSHIKI KAISHA, Tokyo (JP)

(72) Inventor: Yoshio KAWASHIMA, Fujinomiya-shi (JP)

(73) Assignee: TERUMO KABUSHIKI KAISHA, Tokyo (JP)

(21) Appl. No.: 15/428,754

(22) Filed: Feb. 9, 2017

Related U.S. Application Data
(63) Continuation of application No. PCT/JP2015/072614, filed on Aug. 10, 2015.

(30) Foreign Application Priority Data
Aug. 22, 2014 (JP) 2014-169424

(51) Int. Cl. B65B 31/00; B65B 55/16; A61L 2/08; B65D 81/18; B65B 55/08; A61M 25/00; B65B 51/10

(52) U.S. Cl. CPC B65B 31/00; A61M 25/002; B65B 55/16; B65B 51/10; B65D 81/18; B65B 55/08; A61L 2/08; A61L 2202/24; A61L 2202/23

(57) ABSTRACT

A method for manufacturing a packaged medical device includes sealing a packaging bag so that the packaging bag includes a first sub-chamber and a second sub-chamber separated from the first sub-chamber, with the first sub-chamber prevented from communicating with the second sub-chamber. The first sub-chamber contains a medical device and a deoxygenating agent. The first sub-chamber possesses a relatively lower oxygen concentration and the second sub-chamber possesses a relatively higher oxygen concentration. The method also involves communicating the first sub-chamber with the second sub-chamber so that the oxygen concentration in the packaging bag becomes lower than the relatively higher oxygen concentration and higher than the relatively lower oxygen concentration, and irradiating the packaging bag with radiation after the first sub-chamber and the second sub-chamber are in communication with one another.

### CROSS-REFERENCES TO RELATED APPLICATIONS

This application is a continuation of International Application No. PCT/JP2015/072614 filed on Aug. 10, 2015, and claims priority to Japanese Patent Application No. 2014-169424 filed on Aug. 22, 2014, the entire content of both of which is incorporated herein by reference.

### TECHNICAL FIELD

The present invention generally relates to a method for manufacturing a packaged medical device and a packaged medical device. In particular, the invention relates to a method for manufacturing a medical device in a low-oxygen atmosphere.

### BACKGROUND ART

A medical device, such as a catheter, may be made from a material that is affected by oxygen, such as a biological physiological active substance, for example, anticancer drug (hereinafter referred to as a “drug”) and a biodegradable polymer. Examples of such devices include a drug eluting balloon and a drug eluting stent. It is desirable that such medical devices be located in a low-oxygen atmosphere to prevent from being exposed to oxygen as much as possible at the time of transportation or storage. Therefore, the medical device is contained in a packaging bag made from a material that does not permeate oxygen. The inside of the packaging bag is brought into a low-oxygen state (for example, by filling with nitrogen), and then, the packaging bag is sealed.

Medical devices are required to be sterilized after being manufactured to prevent a bacterial infection (i.e., an infection with/caused by bacteria). Examples of a method to sterilize a medical device include methods using heat sterilization, gaseous sterilization, and radiation sterilization including electron beam sterilization. Among these, radiation sterilization can be performed even when the medical device is packaged, thus enabling a simplified/efficient sterilization process. For example, Japanese Patent Application Publication No. H08-89561 discloses hermetically packaging an article including plastic together with a deoxygenating agent and performing electron beam sterilization. This sterilization method enables sterilizing the hermetically packaged article under a low-oxygen atmosphere.

### SUMMARY OF THE INVENTION

As discussed above a medical device (such as a catheter) may be made from a material that is affected by oxygen, such as a drug or a biodegradable polymer. It is thus desirable that, as in the above-mentioned Japanese Patent Application Publication No. H08-89561, the hermetically packaged article be sterilized under a low-oxygen atmosphere. On the other hand, in the case of radiation sterilization (in particular, electron beam sterilization), performing radiation sterilization in an oxygen atmosphere (i.e., a relatively higher-oxygen atmosphere than the low-oxygen atmosphere discussed above) can enhance a sterilization effect. More specifically, when radiation sterilization is performed in an oxygen atmosphere (i.e., a relatively higher oxygen atmosphere), sterilization can be performed with a small dose of radiation (i.e., a relatively smaller dose of radiation). Therefore, the method of performing radiation sterilization under a relatively higher-oxygen atmosphere is higher in sterilization efficiency and more desirable than such a sterilization method as that disclosed in Japanese Patent Application Publication No. H08-89561. Therefore, satisfying both the attainment of a low-oxygen atmosphere in a packaging bag and the improvement in sterilization effect of a medical device was an unresolved problem regarding a method for manufacturing a packaged medical device.

To address this problem, for example, a method could include sealing a packaging bag in which a medical device and a deoxygenating agent are contained in a low-oxygen atmosphere and performing irradiation with radiation immediately after sealing. After that, the method could include lowering the oxygen concentration of the inside of the packaging bag by a deoxygenating agent. The oxygen concentration in the packaging bag during irradiation with radiation could thus be maintained and, after that, the oxygen concentration in the packaging bag could be lowered into a low-oxygen state. In such a method, however, unless time management during a period from sealing of the packaging bag to irradiation with radiation is strictly performed (i.e., irradiation must occur immediately after sealing of the packaging bag), the oxygen concentration in the packaging bag during irradiation with radiation is likely to become too low. Therefore, there are problems with this method because the time management burden during a period from packaging to irradiation with radiation is large (i.e., the time between packaging and irradiation must be very small) and the degree of freedom of a process from manufacturing to irradiation with radiation becomes low.

The method and packaging bag disclosed here address the above-mentioned problems. The disclosed method for manufacturing a packaged medical device is capable of hermetically packaging a medical device in a low-oxygen atmosphere and also temporarily raising the oxygen concentration without requiring the strict controls on time management discussed above.

A method for manufacturing a packaged medical device disclosed here includes a sealing step of preparing a packaging bag having a first sub-chamber and a second sub-chamber that are separated from each other, the first sub-chamber being caused to contain a medical device and a deoxygenating agent and then being sealed, and the second sub-chamber being sealed in a higher oxygen concentration than that in the first sub-chamber, a communication step of causing the first sub-chamber and the second sub-chamber to communicate with each other, and an irradiation step of irradiating the packaging bag with radiation after the communication step.

The method for manufacturing a packaged medical device configured as described above allows for the oxygen concentration in a region in which the medical device is contained to be temporarily raised at optional timing after the sealing step, and the medical device to be placed in a low-oxygen (i.e., relatively lower-oxygen) atmosphere at time points other than the optional timing. Accordingly, the timing of irradiation with radiation can be optionally set (i.e., controlled/determined by the operator) with the medical device contained in the sealed state. Careful time management of the process from manufacturing the medical device to irradiation with radiation is no longer necessary, and the sterilization effect on the medical device by irradiation with radiation can be enhanced. Moreover, the time period that the medical device is located in an oxygen atmosphere (i.e., a relatively higher oxygen atmosphere) can be reduced to a minimum.

The medical device can be configured to have a drug loading portion in which a drug is loaded. The oxygen concentration in the sealed bag temporarily increases in the communication step, but decreases with time due to the deoxygenating agent after the communication step. Therefore, the medical device is located under a low-oxygen atmosphere in the sealing step and for a period from the irradiation step to opening of the packaging bag. Accordingly, the risk of the drug of the medical device decreasing in beneficial effect due to an influence of oxygen, such as oxidation reaction, can be reduced. In other words, the risk of the beneficial effect of a drug decreasing before the medical device is used for a patient can be reduced in the method for manufacturing the medical device.

When a partition portion is formed inside the packaging bag and the first sub-chamber and the second sub-chamber are separated from each other by the partition portion, two spaces that possess a different oxygen concentration can be formed in a single packaging bag (i.e., in the interior of a single bag). Therefore, the packaging bag, in which the first sub-chamber and the second sub-chamber are formed, has a structure that facilitates the communicating the first sub-chamber with the second sub-chamber. For example, breaking the partition portion in the packaging bag enables the first sub-chamber and the second sub-chamber to communicate with each other.

The partition portion may be easily formed by subjecting opposite inner surfaces of the packaging bag to thermal fusion bonding.

The communicating of the first sub-chamber and the second sub-chamber can be facilitated by forming the partition portion in such a manner that the fusion bonding strength of the partition portion is lower than the fusion bonding strength of the peripheral portion of the packaging bag. Pressing the packaging bag with a moderate force thus can enable the partition portion to be relatively easily broken.

When at least a part of the partition portion is broken by pressing the second sub-chamber to cause the first sub-chamber and the second sub-chamber to communicate with each other, the first sub-chamber and the second sub-chamber can be caused to communicate with each other without pressing the first sub-chamber (i.e., in which the medical device is contained). Therefore, the medical device can be prevented from being damaged when the packaging bag is pressed to cause the first and second sub-chambers to communicate with one another.

When radiation used for irradiation in the irradiation step is an electron beam, the medical device can be effectively sterilized under an oxygen atmosphere (i.e., an atmosphere having a relatively higher oxygen concentration). When using electron beam sterilization, for example, heat is hardly generated by electron beam irradiation (i.e., a relatively low amount of heat is generated) and sterilization can be performed in a short time.

When the medical device is a catheter having a drug eluting stent or a drug eluting balloon, the beneficial effect of the drug can be maintained by maintaining the drug applied to the stent or the balloon in a low-oxygen atmosphere at times other than the time of irradiation with radiation,. More specifically, when a catheter having a drug eluting stent or a drug eluting balloon is hermetically packaged, the risk of a drug being deteriorated by, for example, oxidation reaction during storage due to oxygen in the packaging bag can be reduced.

### BRIEF DESCRIPTION OF THE DRAWINGS

FIG. 1 is a plan view of a packaging bag before a partition portion is broken after a medical device is hermetically packaged.

FIG. 2 is a front view of the packaging bag before the partition portion is broken after the medical device is hermetically packaged.

FIG. 3 is a front view of the packaging bag in a state obtained after the partition portion is broken.

FIG. 4 is a plan view of a medical device and a deoxygenating agent inside the packaging bag.

FIG. 5 is a plan view of the packaging bag when a first sub-chamber is formed.

FIG. 6 is a plan view of the packaging bag when a second sub-chamber is formed.

FIG. 7 is a front view illustrating a process proceeding from an increase in oxygen concentration in the packaging bag to sterilization.

FIG. 8 is a graph illustrating the transition of oxygen concentration with time in the packaging bag.

FIG. 9 is a plan view of a packaging bag according to a first modification example.

FIG. 10 is a front view of a packaging bag according to a second modification example.

### MODE FOR CARRYING OUT THE INVENTION

Set forth below with reference to the accompanying drawings is a detailed description of embodiments of a method for manufacturing a packaged medical device (method packaging a medical device) and a packaged medical device representing examples of the inventive method and device disclosed here. Dimensional ratios illustrated in the drawings may be exaggerated for the purpose of illustration and may be different from the actual ratios. In the present specification, the side at which a medical device is inserted into the living body lumen is referred to as a “distal end” or a “distal side”, and the opposite side at which a manipulation is performed (e.g., by a user) is referred to as a “proximal end” or a “proximal side”.

A method for manufacturing a packaged medical device according to an embodiment of this application includes causing a balloon catheter 10 to be hermetically contained in a packaging bag 20. A stent 12 having a drug loading portion (in which a drug is loaded) is mounted on the balloon catheter 10. The method then includes performing sterilization by irradiation with radiation on the balloon catheter 10.

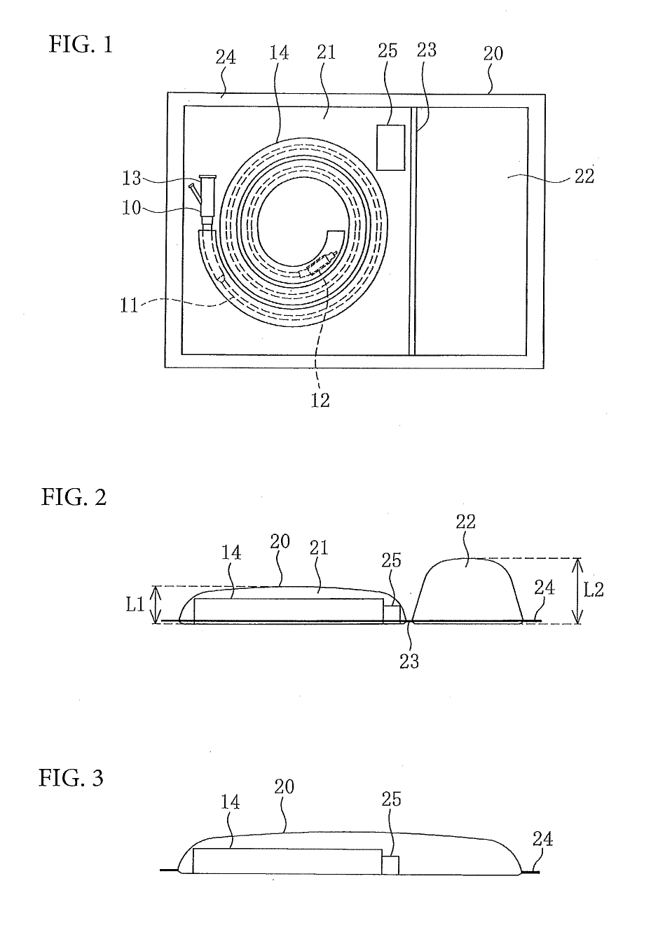

As illustrated in FIG. 1 and FIG. 2, the packaging bag 20 is hermetically sealed in a gas-impermeable state at a peripheral portion 24. The packaging bag 20 is made from a material that does not permeate gas (i.e., is not permeable to gas), such as oxygen. The inside of the packaging bag 20 is partitioned with a partition portion 23 to separate the packaging bag 20 into a first sub-chamber 21 and a second sub-chamber 22. The first sub-chamber 21 and the second sub-chamber 22 are separated from each other in such a way as to prevent circulation of gas between the two chambers 21, 22. The balloon catheter 10 and a deoxygenating agent 25 are contained in the first sub-chamber 21. The inside of the first sub-chamber 21 is filled with nitrogen, and thus has a lower oxygen concentration than that of air. In other words, the balloon catheter 10 is located in a low-oxygen atmosphere inside the packaging bag 20. The inside of the second sub-chamber 22 is filled with a gas that has an oxygen concentration higher than the oxygen concentration inside the first sub-chamber 21.

The packaging bag 20 is hermetically sealed by two film-like sheets being fused and bonded at the peripheral portion 24. The partition portion 23, which is formed between the first sub-chamber 21 and the second sub-chamber 22, is also formed by opposite inner surfaces of the packaging bag 20 being fused and bonded to one another. The first sub-chamber 21 is formed to have a thickness L1 (height) so that the balloon catheter 10 and the deoxygenating agent 25 can be contained between the two sheets (as illustrated in FIG. 2) within the hermetically-sealed packaging bag 20. The second sub-chamber 22 is formed to have a thickness L2 (height). The thickness L2 of the second sub-chamber is greater than that the thickness L1 of the first sub-chamber 21. The second sub-chamber 22 has an increased thickness L2 to increase the volume of the second sub-chamber 22 between the two sheets (as illustrated in FIG. 2) within the hermetically-sealed packaging bag 26.

The partition portion 23 possesses a narrower width than the peripheral portion 24 in such a manner that the fusion bonding strength of the partition portion 23 is lower than the fusion bonding strength of the peripheral portion 24 of the packaging bag 20. The partition portion 23 thus may be able to be broken by pressing of the second sub-chamber 22 at an optional (i.e., desirable) time point after the packaging bag 20 has been sealed. As illustrated in FIG. 3, breaking the partition portion 23 causes the first sub-chamber 21 and the second sub-chamber 22 to communicate with each other, so that the entire packaging bag 20 possesses a single chamber.

As illustrated in FIG. 1, the balloon catheter 10 (which is contained in the packaging bag 20) includes a long and hollow catheter main body portion 11, a balloon provided in proximity to the distal end of the catheter main body portion 11, a stent 12 mounted on an outer circumferential surface of the balloon, and a hub 13 fixed to the proximal end of the catheter main body portion 11. The balloon catheter illustrated in FIG. 1 has an Over-the-Wire structure, but is not limited to this structure. The medical device could be, for example, a balloon catheter having a Rapid Exchange structure.

The balloon catheter 10 is contained inside a holder tube 14. The holder tube 14 is provided to protect the balloon catheter 10 from, for example, contact with or impact from the outside. The holder tube 14 is formed to cover the balloon catheter 10. The holder tube 14 is contained in the packaging bag 20 in a spiral shape as illustrated in FIG. 1.

The balloon catheter 10 includes the hub 13, the catheter main body portion 11, the balloon, and the stent 12 as shown in FIG. 1.

The catheter main body portion 11 is composed of an outer tube and an inner tube located inside the outer tube. The outer is a tube-shaped body possessing an open distal end and an open proximal end. An inflation lumen is formed inside the outer tube. Fluid for inflating the balloon may flow through the inflation lumen. A guide wire lumen through which a guide wire is inserted is formed in the inner tube. The material used to make the inner tube or the outer tube is desirably a material having a certain degree of flexibility. Examples of a flexible material to be used include polyolefin, such as polyethylene, polypropylene, polybutene, ethylene-propylene copolymer, ethylene-vinyl acetate copolymer, ionomer, or a mixture of two or more of these materials, thermoplastic resin, such as soft polyvinyl chloride resin, polyamide, polyamide elastomer, polyester, polyester elastomer, polyurethane, and fluorine resin, silicone rubber, and latex rubber.

The hub 13 includes a first opening portion. An indeflator (a pressure applying device) that communicates with the inflation lumen of the outer tube to supply a fluid for inflating the balloon can be connected to the first opening portion. The hub 13 also includes a second opening portion, which communicates with the guide wire lumen of the inner tube. The fluid for inflating the balloon is, for example, a contrast agent, physiological salt solution, or a mixture of these fluids. Examples of the hub material include thermoplastic resin, such as polycarbonate, polyamide, polysulfone, polyarylate, and methacrylate-butylene-styrene copolymer.

The balloon is mounted in a folded state (or in a deflated state), and is inflated by the above-mentioned fluid being introduced. The inside of the balloon communicates with the inflation lumen of the outer tube. Accordingly, the fluid flows in from the first opening portion of the hub and flows into the inside of the balloon, so that the balloon is inflated. The material used to make the balloon is desirably a flexible material. Examples of a suitable material include a polymer material, such as polyolefin, a cross-linked polymer of polyolefin, polyester, polyester elastomer, polyvinyl chloride, polyurethane, polyurethane elastomer, polyphenylenesulfide, polyamide, polyamide elastomer, and fluorine resin, silicone rubber, and latex rubber. Polyester is, for example, polyethylene terephthalate. The material used to make the balloon is not limited to an embodiment in which the above-mentioned polymer material is solely used, but can be, for example, a film obtained by stacking the above-mentioned polymer materials in layers as appropriate.

The stent 12 keeps a living body lumen at an appropriate size when placed in close contact with the inner surface of a stenosed portion (i.e., a lesion). The stent 12 expands according to the inflation of the balloon. Furthermore, the stent 12 has a drug loading portion, which has a drug, formed on the outer surface of the stent 12.

The stent 12 is made from a biocompatible material. Examples of a biocompatible material include iron, titanium, aluminum, tin, tantalum or a tantalum alloy, platinum or a platinum alloy, gold or a gold alloy, a titanium alloy, a nickel-titanium alloy, a cobalt-base alloy, a cobalt-chrome alloy, stainless steel, a zinc-tungsten alloy, and a niobium alloy. Moreover, the stent can be made from a biodegradable polymer. For example, the biodegradable polymer can be composed of poly(L-lactide), poly(D-lactide), polyglycolide, or poly(L-lactide-co-glycoside).

The drug is coated on the outside surface of the stent 12 and is used to prevent restenosis or reocclusion of the living body lumen, which may occur during placement of the stent 12 at the lesion. In the present embodiment, a mixture of the drug and the biodegradable polymer is used to form the drug loading portion. The drug loading portion is not limited to being provided on the outer surface of the stent, but can be provided on the inner surface of the stent or on both the outer surface and the inner surface of the stent.

The drug is desirably at least one drug selected from the group including, for example, an anticancer drug, immunosuppressive drug, antibiotic, antirheumatic, antithrombogenic drug, HMG-CoA reductase inhibitor, angiotensin-converting enzyme inhibitor, calcium antagonist agent, antihyperlipidemic drug, integrin inhibitor, anti-allergic drug, antioxidant agent, GpIIb/IIIa inhibitor, retinoid, flavonoid, carotenoid, lipid improving drug, DNA synthesis inhibitor, tyrosine kinase inhibitor, antiplatelet drug, anti-inflammatory drug, a tissue-derived biomaterial, interferon, and NO production promoting substance, for the reason that the drug is able to control the behavior of cells of a lesional tissue to treat the lesion. When the drug is immunosuppressive drug and it is rapamycin or a derivative of rapamycin, the effect of preventing restenosis or reocclusion of the living body lumen is increased.

The biodegradable polymer (which is mixed with the drug) only needs to be the one that gradually biologically degrades after the stent 12 is placed at the lesion and has no adverse influence on the living body. Examples of a biodegradable polymer include at least one polymer selected from the group consisting of polyglycolic acid, polylactic acid, polycaprolactone, polyhydroxybutyrate, cellulose, polyhydroxybutyrate valeric acid, and polyorthoester, and a copolymer, a mixture, and a compound of some of these polymers.

The composition ratio (mass ratio) between the drug and the biodegradable polymer can be set to 1:99 to 99:1. But, when the composition ratio of the drug and the biodegradable polymer is between 30:70 and 70:30 (inclusive), as much drug as possible can be loaded while the physical property and separation performance of the biodegradable polymer are acceptable.

A mixture of the drug and the biodegradable polymer can be attached to the surface of the stent main body in the following method. First, the drug and the biodegradable polymer (with the composition ratio such as that described above) are dissolved in a solvent, such as acetone, ethanol, chloroform, or tetrahydrofuran, in such a manner that the solution concentration becomes 0.001% to 20% by mass or, desirably, 0.01% to 10% by mass. This solution is applied to the surface of the stent main body using, for example, a spray or a dispenser. The solvent is volatized, so that a layer of the mixture of the drug and the biodegradable polymer is formed on the surface of the stent. However, the application method described above is an optional method that can be employed, but the application method is not limited to this example.

The drug is able to be present on the surface of the stent main body in an optional embodiment. When a layer is formed on the surface of the stent as a mixture with the biodegradable polymer, however, the drug becomes able to be gradually released in the living body.

The deoxygenating agent 25, which is contained in the packaging bag 20, can be conventionally enclosed and used in the package to preserve a medicinal product or prevent the deactivation or deterioration of a medical product. Examples of such a known deoxygenating agent include an iron-based one and an organic-based oxygen absorber, such as ascorbic acid. While such an oxygen absorber includes a type that absorbs only oxygen, a type that absorbs oxygen and carbon dioxide gas, and a type that absorbs oxygen and generates carbon dioxide gas, any type can be employed.

The deoxygenating agent 25 can be used while retaining an ordinarily-available powder state (i.e., the deoxygenating agent 25 may be employed as a powder that is not packaged in a bag/container), but is desirably used while being packed in, for example, a bag having high permeability to gas. The deoxygenating agent 25 can also be adjusted into a solid-agent form, such as a pill form, or a film-like or sheet-like form.

The size or amount of the deoxygenating agent 25 in any form only needs to be of such a degree to enable the deoxygenating agent 25 to be packed together with the balloon catheter 10 in the first sub-chamber 21 of the packaging bag 20. The number of deoxygenating agents 25 can also be optionally selected as appropriate, but the present embodiment disclosed by way of example includes one enclosure or container containing deoxygenating agent 25.

Although not illustrated, a desiccant agent can also be in the first sub-chamber 21. Using a desiccant agent enables decreasing the possibility of the drug causing a hydrolysis reaction when the medical device has a drug loading portion.

The packaging bag 20 is required to be made using a low gas-permeable substance/material having low permeability to oxygen. The packaging bag 20 is formed from a multi-layered film obtained by stacking a plurality of layers including a low gas-permeable substance. This enables attaining the strength required for the packaging bag 20 and lowering the permeability to oxygen.

A sheet used as a material of the packaging bag 20 in the present embodiment has a multi-layered structure in which a surface layer made from, for example, PET resin or nylon is formed on the outer surface side of an aluminum film. An inner surface layer can be made from, for example, polyethylene. The inner surface layer is formed on the inner surface side of the aluminum film.

An additional layer made from, for example, PET resin or nylon can be formed on the inner surface side of the aluminum film to improve the strength of the sheet. An inner surface layer made from, for example, polyethylene can be formed on the inner surface side of the additional layer. The types or numbers of layers can be changed according to the strength required for the packaging bag 20. It is desirable that the aluminum film be used as an intermediate layer of the sheet of the packaging bag 20 to reliably prevent damage to the aluminum film.

The inner surface layer in the sheet of the packaging bag 20 is made from polyethylene, which has thermoplasticity as mentioned above. Therefore, the inner surface layer can be melted by being heated at about 150° C. to 160° C. At the time of forming the packaging bag 20, peripheral portions of two stacked sheets are heated to melt the inner surface layers, which are then fused and bonded to each other, so that the peripheral portion 24 is formed. The partition portion 23 can also be formed by fusing and bonding the inner surface layers (i.e., portions of the inner surface that are opposite/face one another) to each other in a similar way.

Next, a method of packaging the balloon catheter 10 (i.e., a type of a medical device) is described.

First, a sealing step is performed to prepare the packaging bag 20, which becomes fully sealed after the first sub-chamber 21 and the second sub-chamber 22 are formed therein. The sealing step described here includes causing the balloon catheter 10 to be contained in the packaging bag 20, forming the first sub-chamber 21, forming the second sub-chamber 22, sealing the packaging bag 20, and preparing the sealed packaging bag 20. The sealing step can include purchasing a packaging bag 20 for which the causing the balloon catheter 10 to be contained in the packaging bag 20, the forming of the first sub-chamber 21, the forming of the second sub-chamber 22, and the sealing of the packaging bag 20 are completed, and then the subsequent steps (discussed below) can be performed.

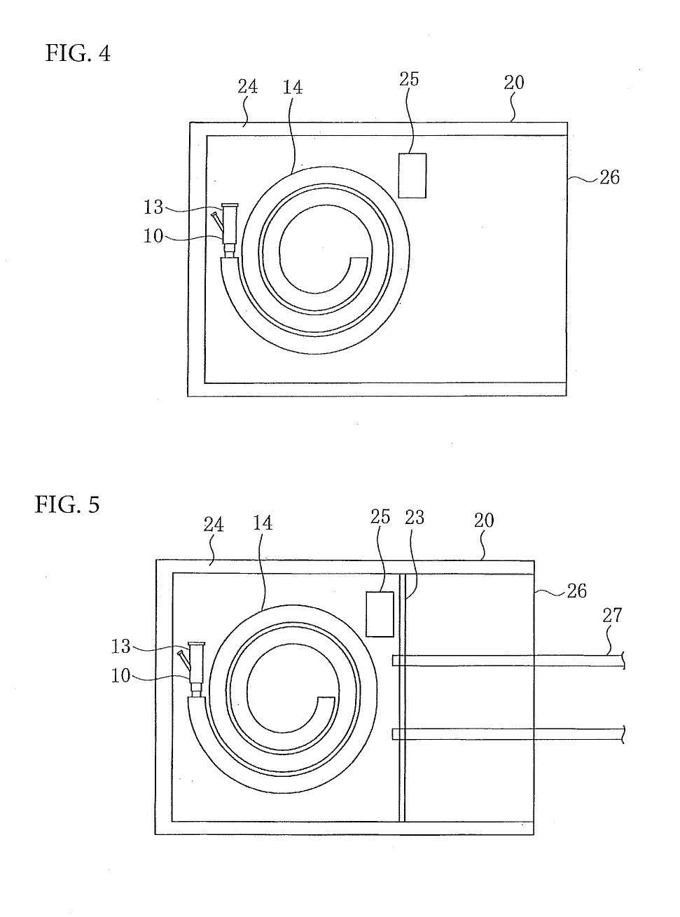

Causing the balloon catheter 10 to be contained in the packaging bag 20, as illustrated in FIG. 4, begins with a packaging bag 20 that has a peripheral portion 24 previously subjected to thermal fusion bonding at three sides of the peripheral portion 24. Only one side of the peripheral portion 24 (used for part of the second sub-chamber 22) is an opening 26 (i.e., remains unsealed). Then, the balloon catheter 10 and the deoxygenating agent 25 are inserted into the packaging bag 20 via the opening 26.

After the balloon catheter 10 and the deoxygenating agent 25 are contained in the packaging bag 20, the partition portion 23 is formed by thermal fusion bonding as illustrated in FIG. 5. At the same time, the first sub-chamber 21 (which is formed by the partition portion 23 and a sealed part of the peripheral portion 24) is filled with nitrogen via a nitrogen injection nozzle 27. Filling the first sub-chamber 21 with nitrogen and forming the partition portion 23 allows the balloon catheter 10 and the deoxygenating agent 25 to be hermetically contained in the first sub-chamber 21 in a nitrogen atmosphere (the stage of forming the first sub-chamber 21).

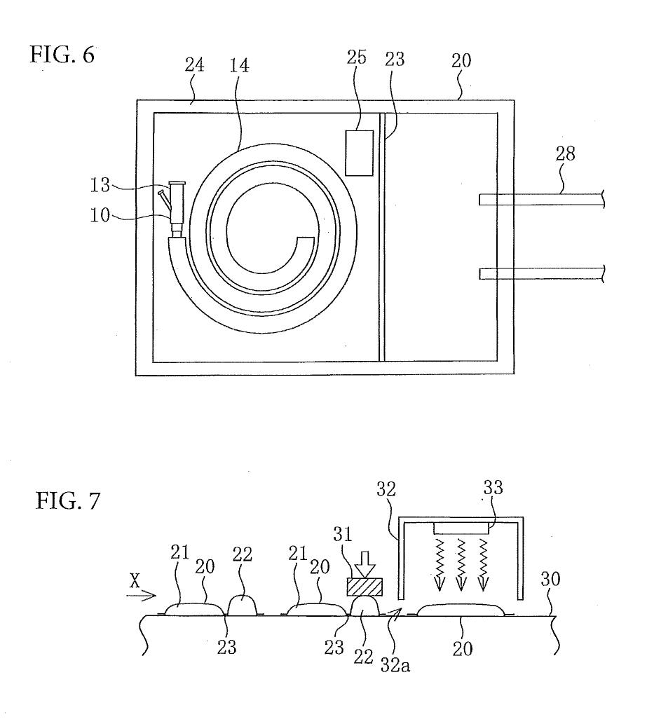

After the first sub-chamber 21 is formed, the peripheral portion 24 is formed at the opening 26 (i.e., the portion of the packaging bag that remained unsealed) by thermal fusion bonding as illustrated in FIG. 6. At the same time, the second sub-chamber 22 (which is formed by the partition portion 23 and the peripheral portion 24) is filled with gas including oxygen by the use of an oxygen injection nozzle 28. Filling the second sub-chamber 22 with oxygen and sealing the rest of the peripheral portion 24 allows the sealed second sub-chamber 22 to enter a state in which the oxygen concentration is higher than the oxygen concentration in the first sub-chamber 21 (the stage of forming the second sub-chamber 22 and the stage of sealing the packaging bag 20).

The size and oxygen concentration of the second sub-chamber 22 relative to the packaging bag 20 can be set as appropriate according to the oxygen concentration needed when the entire packaging bag 20 becomes a single chamber when the partition portion 23 is broken. For example, if the volume of the second sub-chamber 22 is large, the second sub-chamber 22 can be filled with air. Conversely, if the second sub-chamber 22 is filled with pure oxygen, the required volume of the second sub-chamber 22 is relatively smaller. While the second sub-chamber 22 in the present embodiment is formed to have a larger thickness than the first sub-chamber 21, the thickness of the second sub-chamber 22 can be equal to or smaller than that of the first sub-chamber 21 as long as the volume required for the second sub-chamber 22 can be secured.

The procedure of the sealing step is not limited to this example. For example, the method can include previously forming an opening 26 at the side that will be used to form the first sub-chamber 21, filling the second sub-chamber 22 with gas including oxygen and, at the same time, forming the partition portion 23. The method can then include inserting the balloon catheter 10 and the deoxygenating agent 25 via the opening 26, filling the first sub-chamber 21 with nitrogen and, at the same time, forming the peripheral portion 24 at the opening 26 to seal the first sub-chamber 21. In any case, a packaging bag 20 only needs to be prepared with the first sub-chamber 21 and the second sub-chamber 22 that are separated from each other (i.e., the interiors of the first and second sub-chambers 21, 22 are prevented from communicating with each other), the first sub-chamber 21 contains the balloon catheter 10 and the deoxygenating agent 25 and then is sealed, and the second sub-chamber 22 is sealed in a higher oxygen concentration than that in the first sub-chamber 21.

The sealed packaging bag 20 having the first sub-chamber 21 and the second sub-chamber 22 is prepared by the above-described method (the stage of preparing the sealed packaging bag 20). Next, the communication step and the irradiation step are performed. As illustrated in FIG. 7, the sealed packaging bag 20 (in which the balloon catheter 10 is hermetically contained) is loaded on a conveyor belt 30 and is conveyed in the direction X illustrated in the figure. The conveyor belt 30 is provided with pressing means 31. As the pressing means 31, for example, a pressing device can be employed.

To cause the first sub-chamber 21 to communicate with the second sub-chamber 22 (i.e., the interiors of the chambers 21, 22 to communicate), the pressing means 31 presses the second sub-chamber 22 of the packaging bag 20 (i.e., applies a force to the second sub-chamber 22), which has been conveyed there. The pressure within the second sub-chamber 22 is increased by the pressing. Force is thus applied to the thermal-fusing-bonded portions at the four sides of the second-sub chamber 22 that were formed to seal the second sub-chamber 22. The partition portion 23 is narrower in width and lower in fusion bonding strength than the peripheral portion 24, and so only the partition portion 23 is broken by the pressing force of the pressing means 31 (i.e., at least a part of the seal of the partition portion 23 is broken). With this, the first sub-chamber 21 and the second sub-chamber 22 are caused to communicate with each other. Gases in the respective sub-chambers thus mix, and the oxygen concentration in a sealed region containing the balloon catheter 10 becomes higher than before the first sub-chamber 21 and the second sub-chamber 22 were caused to communicate with each other.

In the present embodiment, the second sub-chamber 22 is formed to have a larger thickness L2 (i.e., the thickness between the two sheets) than the first sub-chamber 21. Therefore, the pressing means 31 is able to easily press only the second sub-chamber 22 in a selective manner. The balloon catheter 10 can, for example, be prevented from being damaged by the pressing means 31 because the pressing means 31 does not apply a pressure force to the outer surface of the first sub-chamber 21.

The irradiation step is performed immediately after the communication step. An irradiation region 32 for irradiation with radiation is provided at the downstream side of the pressing means 31 on the conveyor belt 30. The irradiation region 32 includes irradiation means 33. When the packaging bag 20 is conveyed to the irradiation region 32, the packaging bag 20 is irradiated with radiation emitted from the irradiation means 33. The radiation used for irradiation in the irradiation step includes, for example, an electron beam, a gamma ray, and an X-ray. Among these possible examples, an electron beam is desirable because sterilization can be performed in a short time (i.e., relatively more quickly).

When the first sub-chamber 21 and the second sub-chamber 22 have been caused to communicate with each other by the pressing means 31, the packaging bag 20 becomes a single chamber in its entirety. Therefore, the packaging bag 20 becomes smaller in thickness or height than the second sub-chamber 22. Before the irradiation step is performed, it is thus possible to detect whether the partition portion 23 has been broken by the pressing means 31 by discriminating the largeness or smallness in thickness (height) of the packaging bag 20 (e.g., if the packaging bag 20 possesses a relatively larger thickness or height, the packaging bag 20 can be prevented from being conveyed to the irradiation region 32 as discussed below).

Any means for discriminating the thickness of the packaging bag 20 can be employed. For example, an entrance 32a of the irradiation region 32 can be formed to be larger than the thickness of the packaging bag 20 obtained after the partition portion 23 is broken and smaller than the thickness of the second sub-chamber 22 obtained before the partition portion 23 is broken as shown in FIG. 7. Therefore, a packaging bag 20 that is able to pass through the entrance 32a of the irradiation region 32 can be determined to be a packaging bag 20 where the first sub-chamber 21 and the second sub-chamber 22 have been caused to communicate with each other. In other words, the partition portion 23 must have been broken, and so the packaging bag 20 can be advanced to the irradiation step. Conversely, the packaging bag 20 that is unable to pass through the entrance 32a of the irradiation region 32 may have an unbroken partition portion 23 (and so the first sub-chamber 21 and the second sub-chamber 22 do not communicate with each other), and thus can be excluded without being advanced to the irradiation region 32.

The irradiation time of electron beams in the irradiation step needs to be set in such a way to secure the radiation dose required for sterilization of the balloon catheter 10 (i.e., to deliver an appropriate dose for sterilization). The irradiation time is usually set to a value ranging from several seconds to one minute. The radiation dose for irradiation varies with the material or amount of an object to be irradiated, but is usually selected from a range of 2 kGy to 75 kGy.

A sufficient sterilization effect by irradiation with electron beams can be obtained because the oxygen concentration in the region hermetically containing the balloon catheter 10 is raised by causing the first and second sub-chambers 21, 22 to communicate with one another. It is considered that the improved sterilization effect is because oxygen being ozonized or radicalized by irradiation with electron beams enables supplementarily increasing (supplemental increasing of) the sterilization effect of electron beams themselves.

After the first and second sub-chambers 21, 22 have been caused to communicate and the irradiation has been performed, the balloon catheter 10 and the deoxygenating agent 25 are hermetically contained in a single chamber. The oxygen concentration in the packaging bag 20 then gradually decreases due to the deoxygenating agent 25. Eventually, the packaging bag 20 enters a state in which the balloon catheter 10 is hermetically contained in a low-oxygen atmosphere.

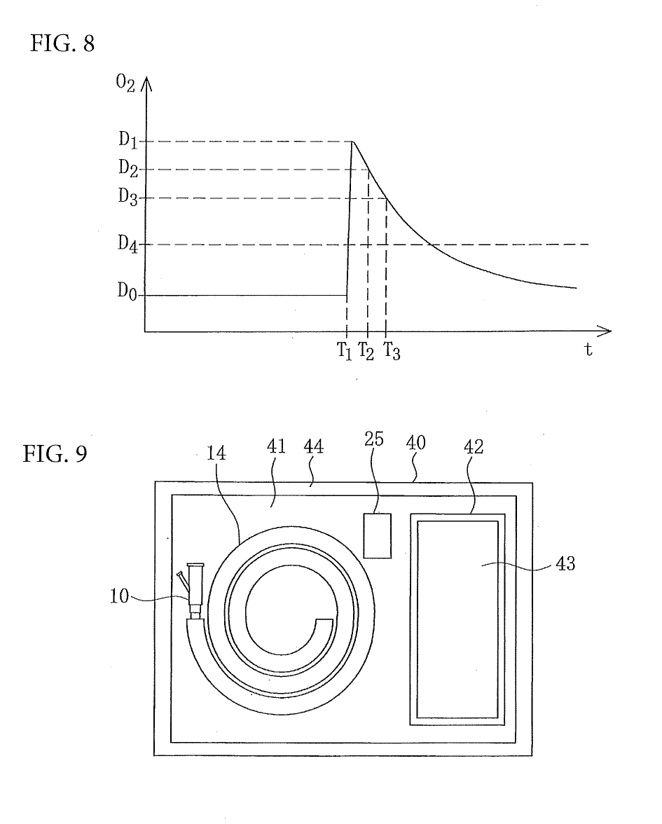

The below description explains how the oxygen concentration in the region in which the balloon catheter 10 is hermetically contained transitions in the communication step and the irradiation step and before and after those steps. In FIG. 8, the abscissa axis (labeled “t”) indicates time, and the ordinate axis (labeled “O₂”) indicates the oxygen concentration in the region in which the balloon catheter 10 is hermetically contained. The minimum oxygen concentration required at the time of irradiation with electron beams is denoted by D₄. The oxygen concentration in the first sub-chamber 21 of the packaging bag 20 before the first sub-chamber 21 communicates with the second sub-chamber 22 is denoted by D₀. D₀ is smaller (i.e., a relatively lower oxygen concentration) than D₄.

Time T₁ is the time at which the communication step is performed. In other words, the time T₁ is when the partition portion 23 is broken. At this time T₁, the first sub-chamber 21 and the second sub-chamber 22 are caused to communicate with each other, so that the oxygen concentration in the region in which the balloon catheter 10 is hermetically contained rapidly increases and becomes D₁. After that, the oxygen concentration in the region in which the balloon catheter 10 is hermetically contained gradually decreases due to the enclosed deoxygenating agent 25.

The irradiation step is performed in a period from time T₂ to time T₃. The oxygen concentration at time T₂ is D₂ and the oxygen concentration at time T₃ is D₃. The oxygen concentrations D₂ and D₃ are each smaller than D₁ at the communication step, but larger than the minimum oxygen concentration D₄ required at the time of irradiation with electron beams. Accordingly, sterilization of the balloon catheter 10 can be efficiently performed by the irradiation step. Since the first sub-chamber 21 and the second sub-chamber 22 are separated from each other and are configured to be able to communicate with each other at an optional time point (e.g., when the partition portion 23 is broken), the communication step can be performed immediately before the irradiation step. Irradiation with electron beams can thus be performed in a state in which the oxygen concentration is sufficiently high.

On the other hand, the balloon catheter 10 can be eventually brought into a state of being hermetically contained in a low-oxygen atmosphere because the oxygen concentration in the packaging bag 20 gradually decreases due to the deoxygenating agent 25 after the irradiation step. Accordingly, a time period for which the drug of the stent 12 is affected by oxygen can be reduced to the minimum.

The below description details a first modification example of the packaging bag and method described above. As illustrated in FIG. 9, a packaging bag 40 of the present modification example is formed as a first sub-chamber 41 in its entirety with a peripheral portion 44 at four sides. The balloon catheter 10 and the deoxygenating agent 25 are contained in the first sub-chamber 41. An inner bag 42 having a second sub-chamber 43 is contained in the first sub-chamber 41. Forming the first sub-chamber 41 and the second sub-chamber 43 with different bags enables bringing the first sub-chamber 41 and the second sub-chamber 43 into a state of being separated from each other without use of the partition portion described above. The sealing of the inner bag 42 creates a partition, however, between the second sub-chamber 43 and the first sub-chamber 41. In the communication step, the inner bag 42 is broken (e.g., by means such as pressing), so that the first sub-chamber 21 and the second sub-chamber 22 communicate with each other.

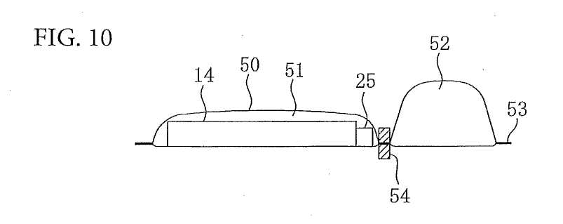

The below description details a second modification example of the packaging bag and method. As illustrated in FIG. 10, a packaging bag 50 of the present modification example is formed to become a single region in its entirety with a peripheral portion 53 at four sides. A sandwich portion 54 is provided at a middle portion of the packaging bag 50. The sandwich portion 54 is formed, for example, in a clip-like shape to be able to pinch and clamp the packaging bag 50 from the outside. The sandwich portion 54 is thus configured to separate a first sub-chamber 51 and a second sub-chamber 52 at opposite sides of the sandwich portion 54. The balloon catheter 10 and the deoxygenating agent 25 are contained within the first sub-chamber 51. The first sub-chamber 51 and the second sub-chamber 52 can be caused to communicate by simply removing the sandwich portion 54.

As described above, the method for manufacturing a packaged medical device according to the present embodiment includes a sealing step of preparing a packaging bag 20 having a first sub-chamber 21 and a second sub-chamber 22 that are separated from each other, the first sub-chamber 21 being caused to contain a medical device and a deoxygenating agent 25 and then being sealed, and the second sub-chamber 22 being sealed in a higher oxygen concentration than that in the first sub-chamber 21, a communication step of causing the first sub-chamber 21 and the second sub-chamber 22 to communicate with each other, and an irradiation step of irradiating the packaging bag 20 with radiation after the communication step, so that, since, in the sealing step, the packaging bag 20 having the first sub-chamber 21 and the second sub-chamber 22 that are separated from each other, the first sub-chamber 21 being caused to contain the medical device and the deoxygenating agent 25 and the second sub-chamber 22 being set to a higher oxygen concentration than that in the first sub-chamber 21, is provided, and the communication step and the irradiation step are performed after the sealing step, the oxygen concentration in a region in which the medical device is contained can be temporarily raised at optional timing after the sealing step, and the medical device can be placed in a low-oxygen atmosphere at time points other than the optional timing. Accordingly, the timing of irradiation with radiation can be optionally set (i.e., controlled by the user) with the medical device contained in the sealed state. Time management of the process from manufacturing of the medical device to irradiation with radiation can be made unnecessary (i.e., the irradiation does not need to occur immediately after manufacturing the medical device). The time period that the medical device is located under an oxygen atmosphere can also be reduced to the minimum.

The medical device can have a drug loading portion in which a drug is loaded. Here, the oxygen concentration in the sealed bag 20 temporarily increases in the communication step, but decreases with time due to the deoxygenating agent 25 after the communication step. The medical device is thus located in a relatively low-oxygen atmosphere in the sealing step and for the time period from the irradiation step to the opening of the packaging bag 20. Accordingly, the risk of the drug of the medical device decreasing in beneficial effect due to an influence of oxygen (such as oxidation reaction) can be reduced. In other words, in the method for manufacturing a packaged medical device according to the present embodiment, the risk of the beneficial effect of a drug decreasing before the medical device is used for a patient can be reduced.

When a partition portion 23 is formed inside the packaging bag 20 to separate the first sub-chamber 21 and the second sub-chamber 22, two spaces different in oxygen concentration can be formed in a single packaging bag 20. Therefore, the packaging bag 20 possesses a structure that facilitates the communication step in the method described in this application. Moreover, since breaking the partition portion 23 in the packaging bag 20 enables the first sub-chamber 21 and the second sub-chamber 22 to communicate with each other, the communication step can be facilitated.

When the partition portion 23 is formed by opposite inner surfaces of the packaging bag 20 being subjected to thermal fusion bonding, the partition portion 23 can be relatively easily formed.

When the partition portion 23 is formed in such a manner that the fusion bonding strength of the partition portion 23 is lower than the fusion bonding strength of the peripheral portion 24 of the packaging bag 20, pressing the packaging bag 20 with a moderate force enables only the partition portion 23 to be easily broken.

When, in the communication step, at least a part of the partition portion 23 is broken by pressing the second sub-chamber 22 to cause the first sub-chamber 21 and the second sub-chamber 22 to communicate with each other, the first sub-chamber 21 and the second sub-chamber 22 can be caused to communicate with each other without pressing the first sub-chamber 21 (in which the balloon catheter 10 is contained). Therefore, the medical device can be prevented from being damaged when the packaging bag 20 is pressed in the communication step in the method disclosed here.

The balloon catheter 10 can also be effectively sterilized under an oxygen atmosphere when radiation used for irradiation in the irradiation step is an electron beam. In the case of electron beam sterilization, for example, heat is hardly generated (i.e., relatively less heat is generated) by electron beam irradiation, and sterilization can be performed in a relatively short time.

When the balloon catheter 10 is a catheter having a drug eluting stent or a drug eluting balloon, since a drug applied to the stent or the balloon is located under a low-oxygen atmosphere at other than the time of irradiation with radiation, a decrease in beneficial effect can be reduced. More specifically, when a catheter having a drug eluting stent or a drug eluting balloon is hermetically packaged, the risk of a drug being deteriorated (e.g., by oxidation reaction) during storage due to oxygen in the packaging bag 20 can be reduced.

The invention is not limited to only the above-described embodiments, but can be changed in various manners by a person skilled in the art within a technical idea of the invention. For example, the medical device contained in the packaging bag 20 can be a medical device other than the above-described balloon catheter 10. Furthermore, the balloon catheter 10 in the present embodiment has a drug eluting stent 12, but can have a drug eluting balloon.

In the above-described embodiment, the fusion bonding strength of the partition portion 23 is relatively low so that only the partition portion 23 is broken (without the peripheral portion 24 being broken) when the second sub-chamber 22 is pressed in the communication step. However, the partition portion 23 can be configured to be broken by other means. For example, the peripheral portion 24 of the packaging bag 20 can be previously sandwiched at four sides by some means, and the second sub-chamber 22 can be pressed to break the partition portion 23. In this case, the strength of the partition portion 23 does not necessarily need to be lower than that of the peripheral portion 24.

Thermal fusion bonding is described above as a method for forming the partition portion 23, but opposite inner surfaces of the packaging bag 20 can be bonded by an adhesive material.

Means for causing the first sub-chamber 21 and the second sub-chamber 22 to communicate with each other in the communication step is also not limited to the pressing means. For example, suctioning both sides of the first sub-chamber 21 and the second sub-chamber 22 across the partition portion 23 can be used to break the partition portion 23.

The detailed description above describes a method for manufacturing a packaged medical device (method of packaging a medical device) and a packaged medical device. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A method for manufacturing a packaged medical device, the method comprising:
sealing a packaging bag so that the packaging bag comprises a first sub-chamber and a second sub-chamber separated from the first sub-chamber, the first sub-chamber being prevented from communicating with the second sub-chamber, the first sub-chamber containing a medical device and a deoxygenating agent, the first sub-chamber possessing a relatively lower oxygen concentration and the second sub-chamber possessing a relatively higher oxygen concentration than the relatively lower oxygen concentration of the first sub-chamber;
communicating the first sub-chamber with the second sub-chamber so that the oxygen concentration in the packaging bag becomes lower than the relatively higher oxygen concentration and higher than the relatively lower oxygen concentration; and
irradiating the packaging bag with radiation after the first sub-chamber and the second sub-chamber are in communication with one another.

2. The method according to claim 1, wherein the medical device comprises a drug loading portion in which a drug is loaded.

3. The method according to claim 1, wherein the sealing of the packaging bag comprises sealing a partition portion to separate the first chamber and the second chamber.

4. The method according to claim 3, wherein
the packaging bag possesses an inner surface, the inner surface comprises a first portion and a second portion opposite the first portion, and
the sealing of the partition portion is formed by thermally fusion bonding the first portion and the second portion of the inner surface to one another.

5. The method according to claim 4, wherein
the sealing of the bag comprises sealing a peripheral portion of the packaging bag, the sealed peripheral portion possessing a fusion bonding strength,
the sealed partition portion possesses a fusion bonding strength, and
the fusion bonding strength of the partition portion is lower than the fusion bonding strength of the peripheral portion of the packaging bag.

6. The method according to claim 3, wherein the enabling of the first and second sub-chambers to communicate comprises breaking at least a part of the partition portion by pressing the second sub-chamber to enable the first sub-chamber and the second sub-chamber to communicate with each other.

7. The method according to claim 1, wherein the radiation used for irradiation is an electron beam.

8. The method according to claim 2, wherein the medical device is a catheter comprising a drug eluting stent or a drug eluting balloon.

9. A method comprising:
partitioning a packaging bag, the packaging bag containing a medical device and a deoxygenating agent, the packaging bag comprising an outer periphery which includes a sealed portion and an unsealed portion, the partitioning creating a first sub-chamber and a second sub-chamber of the medical device, the first and second sub-chambers each possessing an interior, the first sub-chamber being defined by the sealed portion of the outer periphery and the partition, the partitioning preventing the interior of the first sub-chamber from communicating with the interior of the second sub-chamber, the first sub-chamber possessing an oxygen concentration and the second sub-chamber possessing an oxygen concentration, the medical device and the deoxygenating agent being positioned in the first sub-chamber;
causing the interior of the first sub-chamber to possess a relatively lower oxygen concentration; and
sealing the unsealed portion of the outer periphery of the packaging bag to seal the second sub-chamber and cause the second sub-chamber to possess a relatively higher oxygen concentration while the first sub-chamber continues to possess the relatively lower oxygen concentration, the relatively higher oxygen concentration in the second sub-chamber being a higher oxygen concentration than the relatively lower oxygen concentration in the first sub-chamber.

10. The method according to claim 9, wherein the causing of the interior of the first sub-chamber to possess the relatively lower oxygen concentration includes removing oxygen from the interior of the first sub-chamber by the deoxygenating agent.

11. The method according to claim 9, wherein the partitioning creates a partition between the first sub-chamber and the second sub-chamber, and the method further comprises breaking the partition between the first sub-chamber and the second sub-chamber after the sealing of the unsealed portion of the outer periphery of the packaging bag.

12. The method according to claim 11, further comprising irradiating the packaging bag with radiation after the breaking of the partitioning.

13. The method according to claim 9, wherein the partitioning of the plastic bag and the sealing of the unsealed portion is performed by thermal fusion bonding.

14. A medical device packaging bag comprising:
a sealed outer periphery;
a first sub-chamber at least partially surrounded by the sealed outer periphery, the first sub-chamber possessing an interior, the interior of the first sub-chamber possessing a relatively lower oxygen concentration;
a second sub-chamber possessing an interior, the interior of the second sub-chamber possessing a relatively higher oxygen concentration, the relatively higher oxygen concentration in the interior of the second sub-chamber being a higher oxygen concentration than the relatively lower oxygen concentration in the first sub-chamber;
a partition separating the first sub-chamber from the second sub-chamber, the partition preventing the interior of the first sub-chamber from communicating with the interior of the second sub-chamber; and
a medical device located within the interior of the first sub-chamber.

15. The medical device packaging bag according to claim 14, further comprising a deoxygenating agent located within the interior of the first sub-chamber.

16. The medical device packaging bag according to claim 14, wherein
the packaging bag possesses an inner surface, the inner surface comprises a first portion and a second portion opposite the first portion, and
the partition portion is a thermal fusion bonding of the first portion and the second portion of the inner surface to one another

17. The medical device packaging bag according to claim 14, wherein
the sealed outer periphery possesses a fusion bonding strength,
the partition possesses a fusion bonding strength, and
the fusion bonding strength of the partition is lower than the fusion bonding strength of the sealed outer periphery of the packaging bag.

18. The medical device packaging bag according to claim 14, wherein the medical device located within the interior of the first sub-chamber includes a drug loading portion in which a drug is loaded.
